(19) United States
(12) Patent Application Publication
(10) Pub. No.: US 2015/0093313 A1
(43) Pub. Date: Apr. 2, 2015

(54) IONIC LIQUID AND SOLVENT MIXTURES FOR HYDROGEN SULFIDE REMOVAL

(71) Applicant: UOP LLC, Des Plaines, IL (US)

(72) Inventors: Erin M. Broderick, Arlington Heights, IL (US); Alakananda Bhattacharyya, Glen Ellyn, IL (US); Beckay J. Mezza, Arlington Heights, IL (US)

(73) Assignee: UOP LLC, Des Plaines, IL (US)

(21) Appl. No.: 14/042,647

(22) Filed: Sep. 30, 2013

(51) Int. Cl. B01D 53/52; C07C 7/11

(52) U.S. Cl. CPC B01D 53/526; C07C 7/11
USPC 423/226; 423/220; 585/866

(57) ABSTRACT

The invention comprises a process for removal of hydrogen sulfide from gaseous mixtures. The process involves the use of a mixture of a physical absorption solvent and an ionic liquid. The mixtures provided improved absorption of hydrogen sulfide, when compared to physical absorption solvents without the ionic liquid at low partial pressures of hydrogen sulfide. A regeneration cycle involving the addition of a solvent, such as water, is used to regenerate the mixture.

### BACKGROUND OF THE INVENTION

The separation of hydrogen sulfide from gas mixtures, such as natural gas, flue gas, syngas and shale gas, is of industrial importance. The removal of hydrogen sulfide is necessary to improve the fuel quality of the natural gas or to use syngas. Its removal is important for environmental reasons both because it is a poisonous gas in sufficient quantities but also because when heated it reacts to form sulfur dioxide, which is also an environmental hazard. In addition, hydrogen sulfide can be corrosive to metal pipes, which makes the removal of hydrogen sulfide necessary for transportation of natural gas. Hydrogen sulfide is an acid gas that is a contaminant of natural gas. Current absorption solvents, such as aqueous amines, including alkanolamines, and dimethyl ethers of polyethylene glycol, are capable of absorbing H₂S, but have disadvantages. Regeneration and decomposition of the amines can be problematic in the presence of H₂S. A higher capacity for hydrogen sulfide and a milder regeneration cycle in comparison to aqueous amines is desired. Common issues with physical absorption solvents are low operating temperatures and high operating pressures. In addition, effluent washes may be needed to recover solvents lost in the feed stream.

Ionic liquids are capable of solubilizing or reacting with polar molecules. Ionic liquids are comprised of a cation and anion and are liquid at or below the process temperature. Ionic liquids characteristically are non-flammable, non-degradable, viscous, thermally stable and have a low vapor pressure. Many of these characteristics are properties that are preferred in connection with solutions to the problems of current hydrogen sulfide removal technology. While many of the characteristics of ionic liquids are beneficial, the high viscosity of ionic liquids may provide challenges to their use. It has now been found that ionic liquids can be added to absorption solvents in a variety of weight percents to alleviate the viscosity issue, and achieve results that are better than the use of a solvent without the ionic liquid. Imidazolium and phosphonium based ionic liquids are added to physical absorption solvents at a range of concentrations, which impact the performance of the ionic liquid. Improvements have been found in the removal of hydrogen sulfide through the use of these combinations of ionic liquids and physical absorption solvents.

### SUMMARY OF THE INVENTION

The invention involves a process for removing hydrogen sulfide from a gas stream comprising contacting the gas stream with a mixture of an ionic liquid and a physical absorption solvent. The physical absorption solvent may be selected from, but is not limited to, the group consisting of dimethyl ether of propylene glycol (DEPG), N-methyl-2-pyrrolidone, methanol, propylene carbonate, poly(propylene glycol) di-methyl ether (PPGDME), poly(propylene glycol) di-acetate (PPGDAc), poly(butylene glycol) di-acetate (PBGDAc) with linear or branched C4 monomers, poly(dimethyl siloxane) (PDMS), perfluoropolyether (PFPE), glycerol tri-acetate (GTA), acetone, methyl acetate, 1,4-dioxane, 2-methoxyethyl acetate, 2-nitropropane, n,n-dimethylacetamide, acetylacetone, 1-nitropropane, isooctane, 2-(2-butoxyethoxy)ethyl acetate, n-formylmorpholine, 2-butoxyethyl acetate, and n-tert-butylformamide.

Preferably, the physical absorption solvent is selected from the group consisting of dimethyl ether of propylene glycol, N-methyl-2-pyrrolidone, methanol and propylene carbonate.

The ionic liquid comprises a cation selected from, but is not limited to, the group consisting of ammonium, phosphonium, imidazolium, pyrazolium, pyridinium, pyrrolidinium, sulfonium, piperidinium, caprolactamium, guanidinium, and morpholium. The ionic liquid comprises an anion selected from the group consisting of halides, carboxylates, sulfonates, sulfates, tosylates, carbonates, phosphates, phosphinates, borates, cyanates, bis(trifluoromethylsulfonyl) imides, and aprotic heterocyclic anions. Preferably, the cation is an imidazolium or a tetraalkyl phosphonium and the anion is an acetate. The mixture may comprise from about 1-99 vol % ionic liquid and from about 1-99 vol % physical absorption solvent, from about 5-95 vol % ionic liquid and from about 5-95 vol % physical absorption solvent, from about 25-75 vol % ionic liquid and from about 25-75 vol % physical absorption solvent or from about 40-60 vol % ionic liquid and from about 40-60 vol % physical absorption solvent. The physical absorption solvent may be a nonprotic solvent or a protic solvent. The process may further comprise regeneration of the mixture of ionic liquid and physical absorption solvent. The regeneration of the absorbed hydrogen sulfide may first comprise the addition of a solvent to remove hydrogen sulfide from the mixture. The resulting mixture of ionic liquid, physical absorption solvent and regeneration solvent may have heat applied to remove volatiles.

Another embodiment of the regeneration of the mixture of solvent and ionic liquid where the physical absorption solvent and ionic liquid chemically absorbed the carbon dioxide and hydrogen sulfide comprises first sending a mixture of physical absorption solvent and ionic liquid to a pressure swing adsorption bed to remove carbon dioxide, followed by adding a solvent to remove hydrogen sulfide and then applying heat to remove the volatiles.

### DESCRIPTION OF THE INVENTION

One embodiment of the invention involves a composition comprising an ionic liquid and a physical absorption solvent. The physical absorption solvents that may be used include, but are not limited to, dimethyl ethers of propylene glycol (DEPG), N-methyl-2-pyrrolidone, methanol, propylene carbonate, poly(propylene glycol) di-methyl ether (PPGDME), poly(propylene glycol) di-acetate (PPGDAc), poly(butylene glycol) di-acetate (PBGDAc) with linear or branched C₄ monomers, poly(dimethyl siloxane) (PDMS), perfluoropolyether (PFPE), glycerol tri-acetate (GTA), acetone, methyl acetate, 1,4-dioxane, 2-methoxyethyl acetate, 2-nitropropane, n,n-dimethylacetamide, acetylacetone, 1-nitropropane, isooctane, 2-(2-butoxyethoxy)ethyl acetate, n-formylmorpholine, 2-butoxyethyl acetate, and n-tert-butylformamide. Preferably, the physical absorption solvent is a dimethyl ether of propylene glycol, N-methyl-2-pyrrolidone, methanol and propylene carbonate. The cation of the ionic liquids may be selected from, but is not limited to, the following: ammonium, phosphonium, imidazolium, pyrazolium, pyridinium, pyrrolidinium, sulfonium, piperidinium, caprolactamium, guanidinium and morpholium. Phosphonium and imidazolium ionic liquids are preferred. The anion of the ionic liquid may be selected from, but is not limited to, the following: halides, carboxylates, sulfonates, sulfates, tosylates, carbonates, phosphates, phosphinates, borates, cyanates, bis(trifluoromethylsulfonyl)imides, and aprotic heterocyclic anions. The ionic liquid is preferably selected from the group consisting of phosphonium and imidazolium acetate ionic liquids. The composition may further comprise water.

The composition may comprise about 1-99 vol % ionic liquid and about 1-99 vol % physical absorption solvent. It may comprise about 5-95 vol % ionic liquid and about 5-95 vol % physical absorption solvent. In other embodiments, the composition comprises about 25-75 vol % of the ionic liquid and about 25-75 vol % of the physical absorption solvent. In another embodiment of the invention, the composition comprises about 40-60 vol % of the ionic liquid and about 40-60 vol % of the physical absorption solvent.

The invention also comprises the method of purifying gas streams that are also referred to as gaseous mixtures by use of these compositions. This method comprises contacting a gas mixture with a mixture of an ionic liquid and a physical absorption solvent in an absorbent zone wherein the ionic liquid and physical absorption solvent mixture absorbs at least one component from said gas mixture, and then the ionic liquid and physical absorption solvent mixture is regenerated to remove the absorbed component or components. The method is useful for sulfur containing gas mixtures and in particular hydrogen sulfide containing gas mixtures. Among the gas mixtures that may be treated are natural gas, flue gas, syngas, and shale gas.

In the method, the physical absorption solvents that may be used include, but are not limited to, dimethyl ethers of propylene glycol (DEPG), N-methyl-2-pyrrolidone, methanol, propylene carbonate, poly(propylene glycol) di-methyl ether (PPGDME), poly(propylene glycol) di-acetate (PPGDAc), poly(butylene glycol) di-acetate (PBGDAc) with linear or branched C4 monomers, poly(dimethyl siloxane) (PDMS), perfluoropolyether (PFPE), glycerol tri-acetate (GTA), acetone, methyl acetate, 1,4-dioxane, 2-methoxyethyl acetate, 2-nitropropane, n,n-dimethylacetamide, acetylacetone, 1-nitropropane, isooctane, 2-(2-butoxyethoxy)ethyl acetate, n-formylmorpholine, 2-butoxyethyl acetate, and n-tert-butylformamide. Preferably, the physical absorption solvent is dimethyl ethers of propylene glycol, N-methyl-2-pyrrolidone, methanol and propylene carbonate. The cation of the ionic liquids may be selected from, but is not limited to, the following: ammonium, phosphonium, imidazolium, pyrazolium, pyridinium, pyrrolidinium, sulfonium, piperidinium, caprolactamium, guanidinium and morpholium. The anion of the ionic liquid may be selected from, but is not limited to, the following: halides, carboxylates, sulfonates, sulfates, tosylates, carbonates, phosphates, phosphinates, borates, cyanates, bis(trifluoromethylsulfonyl)imides, and aprotic heterocyclic anions. The preferred ionic liquids may be selected from the group consisting of phosphonium and imidazolium acetate ionic liquids. The composition may further comprise water. The mixture of physical absorption solvent and ionic liquid may comprise from about 5-95 vol % ionic liquid and from about 5-95 vol % physical absorption solvent. In another embodiment, the mixture comprises from about 25-75 vol % of the ionic liquid and from about 25-75 vol % of the physical absorption solvent. The mixture may comprise from about 40-60 vol % of said ionic liquid and from about 40-60 vol % of said physical absorption solvent. The method is particularly useful for gas mixtures containing carbon dioxide. Among the gas mixtures that may be treated are natural gas, flue gas, synthesis gas, and shale gas. Natural gas is a naturally occurring hydrocarbon gas consisting mainly of methane, varying amounts of higher carbon alkanes, carbon dioxide, nitrogen, and hydrogen sulfide as well as impurities. Synthesis gas is a fuel gas mixture consisting primarily of hydrogen, carbon monoxide and often carbon dioxide as well as impurities. Shale gas is a form of natural gas that is found trapped within shale formations and is an increasingly important source of natural gas. Flue gas is the gas exiting a furnace or power plant and consists of nitrogen, carbon dioxide, water vapor, oxygen, and pollutants such as soot, carbon monoxide, nitrogen oxides and sulfur oxides.

The addition of an ionic liquid to a physical absorption solvent has the capability to eliminate the need for refrigeration and effluent washing. The addition of ionic liquids to physical absorption solvents at a range of concentrations demonstrates an increase in performance compared to the physical absorption solvents at low partial pressures of H₂S. Among the benefits of ionic liquid addition to physical absorption solvent are an increased performance in capacity and a lower possible operating pressure.

In the present invention, a physical absorption solvent is added to a phosphonium or imidazolium based ionic liquid and stirred until a homogenous mixture results. The ionic liquid and solvent mixture was placed in an autoclave that was pressurized with 5515 kPa (800 psi) of a 1 mol % H₂S/CH₄ gas mixture. The ionic liquid and solvent mixture was stirred for 1 hour at room temperature, and then a sample from the gas headspace was taken and analyzed by gas chromatography for hydrogen sulfide content (Table 1). It is noted that liters accounts for all liquid (for example IL+solvent).

TABLE 1

| | | wt % | Capacity |
|---|---|---|---|
| Ionic Liquid | Solvent | Solvent | (mol H₂S/L) |
| bmimOAc | none | 0 | 0.413 |
| bmimOAc | DEPG | 17 | 0.35 |
| bmimOAc | DEPG | 51 | 0.36 |
| bmimOAc | DEPG | 74 | 0.148 |
| bmimOAc | DEPG | 83 | not detected |
| none | DEPG | 100 | not detected |
| bmimOAc | MeOH | 49 | not detected |
| none | MeOH | 100 | not detected |

Table 1 shows the results with 1-butyl-3-methylimidazolium acetate ionic liquid and the percentages shown of the physical solvents. Table 2 compares carbon dioxide and hydrogen sulfide absorption with 55 kPa (8 psi) of carbon dioxide or hydrogen sulfide acid gas added. When the absorption of H₂S was compared to the absorption of CO₂, it was observed that depending on the added solvent, the ionic liquid material selectively absorbed H₂S. The ionic liquids used in the following examples are tris(butyl/propyl)methylphosphonium acetate (abbreviated as PmixOAc) and 1-butyl-3-methylimidazolium acetate (abbreviated as bmimOAc).

A part of the process of the invention is the regeneration of the ionic liquid/physical absorption solvent mixtures. In one embodiment, a mixture of methane and hydrogen sulfide contacts a mixture of ionic liquid and physical absorption solvent and hydrogen sulfide is absorbed by the ionic liquid mixture resulting in a separate methane product flow. A solvent, such as water, alcohols, alkanes, ethers or aromatic solvents is then added to the mixture so that hydrogen sulfide will be released for removal. Then heat can be applied to remove the solvent. The remaining ionic liquid/solvent mixture then can be recycled to remove hydrogen sulfide.

TABLE 2

| | | wt % | Capacity | Capacity |
|---|---|---|---|---|
| Ionic Liquid | Solvent | Solvent | (mol H₂S/L) | (mol CO₂/L) |
| PmixOAc | MeOH | 75 | not detected | 0.13 |
| PmixOAc | DEPG | 74 | 0.24 | 0.26 |

In another embodiment, the stream that is being treated is a mixture of methane, carbon dioxide and hydrogen sulfide which is contacted with a mixture of ionic liquid and a solvent. The CO₂ and H₂S may be removed by this step depending upon the amount and type of solvent added to the ionic liquid. Then there is a two step regeneration with a pressure swing adsorption bed that is operated under conditions sufficient to cause the carbon dioxide to be desorbed and a mixture of ionic liquid, solvent and hydrogen sulfide passes through for further treatment. Then a solvent, such as water, is added as above to cause the hydrogen sulfide to be removed. The added solvent, such as water, is then removed.

The operating conditions included 5515 kPa (800 psi) of 1 mol % H₂S/CH₄ mixture added to an autoclave and stirred for 1 hour at room temperature. Further additions of H₂S to the ionic liquid and solvent mixture, resulted in a decrease in capacity (Table 3).

TABLE 3

| | | Capacity |
|---|---|---|
| Ionic Liquid | H₂S added | (mol H₂S/L) |
| PmixOAc | 55 kPa (8 psi) | 0.384 |
| | 55 kPa (8 psi) | 0.414 |
| | 55 kPa (8 psi) | 0.008 |
| regen with water | 55 kPa (8 psi) | 0.417 |

For regeneration, a solvent, for example water, was added to the ionic liquid phase and stirred. The chemisorbed H₂S was desorbed, and the added solvent (water) was removed from the ionic liquid with heat and reduced pressure. The ionic liquid was then retested for hydrogen sulfide absorption (Table 4). Other solvents, such as but not limited to methanol, acetone, and pentane may be added during the regeneration process to aid in the desorption of H₂S.

TABLE 4

| | Capacity |
|---|---|
| Ionic Liquid | (mol H₂S/L) |
| bmimOAc | 0.326 |
| regen 1 | 0.358 |
| regen 2 | 0.335 |
| regen 2 | 0.374 |

Based upon these experiments, it was found that mixtures of ionic liquids+solvents (both non-aqueous and aqueous) are capable of absorbing hydrogen sulfide from a methane/hydrogen sulfide mixture. Increasing the wt % of solvent in the mixture decreases the hydrogen sulfide absorption capacity. When equal amounts (based on wt %) of a protic solvent (such as methanol) and non-protic solvent (such as dimethylethers of polyethylene glycol) are added to an ionic liquid, the ionic liquid and non-protic solvent mixture has a greater capacity for hydrogen sulfide absorption than the ionic liquid and protic solvent mixture. Carbon dioxide can be absorbed by some ionic liquid+solvent mixtures that absorb very little hydrogen sulfide. Depending upon the operation of the process, it is possible to absorb hydrogen sulfide or carbon dioxide or both, as needed from a mixture with methane. The capacity of an ionic liquid for hydrogen sulfide absorption decreases with further addition of hydrogen sulfide, but the ionic liquid that has chemisorbed hydrogen sulfide can be regenerated through the addition of solvent, such as water. The ionic liquid that has chemisorbed hydrogen sulfide can be regenerated at least several times.

The absorption of hydrogen sulfide can be suppressed in an ionic liquid and solvent mixture through the addition of a protic solvent. Protic solvent and IL mixtures that absorb very little hydrogen sulfide are still capable of absorbing carbon dioxide. Ionic liquids that chemically absorbed hydrogen sulfide can be regenerated through solvent addition.

### EXAMPLES

### H₂S Absorption

Methanol (0.057 g, 0.018 mol) was added to tri(butyl/propyl)methylphosphonium acetate (2.93, 0.010 mol) and the mixture was stirred until a homogeneous mixture resulted. The mixture was loaded into a 70 mL autoclave and pressurized with a 5515 kPa (800 psi) H₂S (1%)/CH₄ gas mixture. After stirring for an hour at room temperature, a GC of the headspace was taken and a decrease in sulfur content was observed.

### Regeneration of Ionic Liquid

Water (20 g) was added to a tri(butyl/propyl)methylphosphonium acetate and methanol (15 wt %) mixture (3.45 g). Volatiles were removed through heating (100° C.) under reduced pressure. Methanol was added again (15 wt %) to the ionic liquid, and the H₂S absorption experiment was repeated.

### SPECIFIC EMBODIMENTS

While the following is described in conjunction with specific embodiments, it will be understood that this description is intended to illustrate and not limit the scope of the preceding description and the appended claims.

A first embodiment of the invention is a process for removing hydrogen sulfide from a gas stream comprising contacting the gas stream with a mixture of an ionic liquid and a physical absorption solvent or a non-aqueous solvent. An embodiment of the invention is one, any or all of prior embodiments in this paragraph up through the first embodiment in this paragraph wherein the gas stream is selected from the group consisting of natural gas, flue gas, synthesis gas and shale gas. An embodiment of the invention is one, any or all of prior embodiments in this paragraph up through the first embodiment in this paragraph, wherein the physical absorption solvent is selected from the group consisting of dimethyl ether of propylene glycol (DEPG), N-methyl-2-pyrrolidone, methanol, propylene carbonate, poly(propylene glycol) di-methyl ether (PPGDME), poly(propylene glycol) di-acetate (PPGDAc), poly(butylene glycol) di-acetate (PBGDAc) with linear or branched C4 monomers, poly(dimethyl siloxane) (PDMS), perfluoropolyether (PFPE), glycerol tri-acetate (GTA), acetone, methyl acetate, 1,4-dioxane, 2-methoxyethyl acetate, 2-nitropropane, n,n-dimethylacetamide, acetylacetone, 1-nitropropane, isooctane, 2-(2-butoxyethoxy)ethyl acetate, n-formylmorpholine, 2-butoxyethyl acetate, and n-tert-butylformamide. An embodiment of the invention is one, any or all of prior embodiments in this paragraph up through the first embodiment in this paragraph wherein the non-aqueous solvent is selected from the group consisting of alkanes, alkenes, aromatics, ethers, alcohols, ketones, and polar aprotics. An embodiment of the invention is one, any or all of prior embodiments in this paragraph up through the first embodiment in this paragraph wherein the alkanes are selected from the group consisting of pentane, hexane, heptane, octane, and cyclohexane, the alkenes are selected from the group consisting of butene and pentene, the aromatics are selected from the group consisting of toluene, benzene, and xylene, the ethers are selected from the group consisting of dimethyl ether, diethyl ether, and tetrahydrofuran, the alcohols are selected from the group consisting of ethanol, isopropanol, butanol, pentanol, hexanol, heptanol, propylene glycol, ethylene glycol, and glycerol, the ketones are selected from the group consisting of acetone, butanone, and 3-pentanone, and the polar aprotics are selected from the group consisting of dichloromethane, acetonitrile, chloroform, dimethylformamide, and dimethylsulfoxide. An embodiment of the invention is one, any or all of prior embodiments in this paragraph up through the first embodiment in this paragraph wherein the physical absorption solvent is selected from the group consisting of dimethyl ether of propylene glycol (DEPG), N-methyl-2-pyrrolidone, methanol and propylene carbonate. An embodiment of the invention is one, any or all of prior embodiments in this paragraph up through the first embodiment in this paragraph wherein the ionic liquid comprises a cation selected from the group consisting of ammonium, phosphonium, imidazolium, pyrazolium, pyridinium, pyrrolidinium, sulfonium, piperidinium, caprolactamium, guanidinium, and morpholium. An embodiment of the invention is one, any or all of prior embodiments in this paragraph up through the first embodiment in this paragraph wherein the ionic liquid comprises an anion selected from the group consisting of halides, carboxylates, sulfonates, sulfates, tosylates, carbonates, phosphates, phosphinates, borates, cyanates, bis(trifluoromethylsulfonyl) imides, and aprotic heterocyclic anions. An embodiment of the invention is one, any or all of prior embodiments in this paragraph up through the first embodiment in this paragraph wherein the cation is an imidazolium or a tetraalkyl phosphonium. An embodiment of the invention is one, any or all of prior embodiments in this paragraph up through the first embodiment in this paragraph wherein the anion is an acetate. An embodiment of the invention is one, any or all of prior embodiments in this paragraph up through the first embodiment in this paragraph wherein the composition comprises from about 1-99 vol % ionic liquid and from about 1-99 vol % physical absorption solvent. An embodiment of the invention is one, any or all of prior embodiments in this paragraph up through the first embodiment in this paragraph wherein the composition comprises from about 5-95 vol % ionic liquid and from about 5-95 vol % physical absorption solvent. An embodiment of the invention is one, any or all of prior embodiments in this paragraph up through the first embodiment in this paragraph wherein the composition comprises from about 25-75 vol % of the ionic liquid and from about 25-75 vol % of the physical absorption solvent. An embodiment of the invention is one, any or all of prior embodiments in this paragraph up through the first embodiment in this paragraph wherein the composition comprises from about 40-60 vol % of the ionic liquid and from about 40-60 vol % of the physical absorption solvent. An embodiment of the invention is one, any or all of prior embodiments in this paragraph up through the first embodiment in this paragraph wherein the physical absorption solvent is a nonprotic solvent or a protic solvent. An embodiment of the invention is one, any or all of prior embodiments in this paragraph up through the first embodiment in this paragraph further comprises regeneration of the mixture of ionic liquid and physical absorption solvent wherein the regeneration first comprises addition of a solvent to remove hydrogen sulfide from the mixture and then a resulting mixture of ionic liquid, physical absorption solvent and regeneration solvent is heated and fractionated to separate the volatiles. An embodiment of the invention is one, any or all of prior embodiments in this paragraph up through the first embodiment in this paragraph further comprising regenerating the ionic liquid that is loaded with carbon dioxide and hydrogen sulfide by first sending the ionic liquid through a pressure swing adsorber to remove carbon dioxide followed by addition of a solvent to remove the hydrogen sulfide. An embodiment of the invention is one, any or all of prior embodiments in this paragraph up through the first embodiment in this paragraph wherein the solvent is selected from the group consisting of water, alcohols, alkanes, alkenes, ethers, ketones, polar aprotic and aromatic solvents. An embodiment of the invention is one, any or all of prior embodiments in this paragraph up through the first embodiment in this paragraph wherein the process further comprises addition of a protic or a non-protic solvent to the ionic liquid. An embodiment of the invention is one, any or all of prior embodiments in this paragraph up through the first embodiment in this paragraph wherein the operating temperature is between 0° C. and 100° C. and operating pressure is between 689 kPa (100 psi) and 14 MPa (2000 psi).

1. A process for removing hydrogen sulfide from a gas stream comprising contacting the gas stream with a mixture of an ionic liquid and a physical absorption solvent or a non-aqueous solvent.

2. The process of claim 1 wherein said gas stream is selected from the group consisting of natural gas, flue gas, synthesis gas and shale gas.

3. The process of claim 1 wherein the physical absorption solvent is selected from the group consisting of dimethyl ether of propylene glycol (DEPG), N-methyl-2-pyrrolidone, methanol, propylene carbonate, poly(propylene glycol) di-methyl ether (PPGDME), poly(propylene glycol) di-acetate (PPGDAc), poly(butylene glycol) di-acetate (PBGDAc) with linear or branched C₄ monomers, poly(dimethyl siloxane) (PDMS), perfluoropolyether (PFPE), glycerol tri-acetate (GTA), acetone, methyl acetate, 1,4-dioxane, 2-methoxyethyl acetate, 2-nitropropane, n,n-dimethylacetamide, acetylacetone, 1-nitropropane, isooctane, 2-(2-butoxyethoxy)ethyl acetate, n-formylmorpholine, 2-butoxyethyl acetate, and n-tert-butylformamide.

4. The process of claim 1 wherein the non-aqueous solvent is selected from the group consisting of alkanes, alkenes, aromatics, ethers, alcohols, ketones, and polar aprotics.

5. The process of claim 4 wherein the alkanes are selected from the group consisting of pentane, hexane, heptane, octane, and cyclohexane, the alkenes are selected from the group consisting of butene and pentene, the aromatics are selected from the group consisting of toluene, benzene, and xylene, the ethers are selected from the group consisting of dimethyl ether, diethyl ether, and tetrahydrofuran, the alcohols are selected from the group consisting of ethanol, isopropanol, butanol, pentanol, hexanol, heptanol, propylene glycol, ethylene glycol, and glycerol, the ketones are selected from the group consisting of acetone, butanone, and 3-pentanone, and the polar aprotics are selected from the group consisting of dichloromethane, acetonitrile, chloroform, dimethylformamide, and dimethylsulfoxide.

6. The process of claim 1 wherein said physical absorption solvent is selected from the group consisting of dimethyl ether of propylene glycol (DEPG), N-methyl-2-pyrrolidone, methanol and propylene carbonate.

7. The process of claim 1 wherein said ionic liquid comprises a cation selected from the group consisting of ammonium, phosphonium, imidazolium, pyrazolium, pyridinium, pyrrolidinium, sulfonium, piperidinium, caprolactamium, guanidinium, and morpholium.

8. The process of claim 1 wherein said ionic liquid comprises an anion selected from the group consisting of halides, carboxylates, sulfonates, sulfates, tosylates, carbonates, phosphates, phosphinates, borates, cyanates, bis(trifluoromethylsulfonyl) imides, and aprotic heterocyclic anions.

9. The process of claim 7 wherein said cation is an imidazolium or a tetraalkyl phosphonium.

10. The process of claim 8 wherein said anion is an acetate.

11. The process of claim 1 wherein said composition comprises from about 1-99 vol % ionic liquid and from about 1-99 vol % physical absorption solvent.

12. The process of claim 1 wherein said composition comprises from about 5-95 vol % ionic liquid and from about 5-95 vol % physical absorption solvent.

13. The process of claim 1 wherein said composition comprises from about 25-75 vol % of said ionic liquid and from about 25-75 vol % of said physical absorption solvent.

14. The process of claim 1 wherein said composition comprises from about 40-60 vol % of said ionic liquid and from about 40-60 vol % of said physical absorption solvent.

15. The process of claim 1 wherein said physical absorption solvent is a nonprotic solvent or a protic solvent.

16. The process of claim 1 further comprises regeneration of said mixture of ionic liquid and physical absorption solvent wherein said regeneration first comprises addition of a solvent to remove hydrogen sulfide from said mixture and then a resulting mixture of ionic liquid, physical absorption solvent and regeneration solvent is heated and fractionated to separate the volatiles.

17. The process of claim 1 further comprising regenerating the ionic liquid that is loaded with carbon dioxide and hydrogen sulfide by first sending said ionic liquid through a pressure swing adsorber to remove carbon dioxide followed by addition of a solvent to remove said hydrogen sulfide.

18. The process of claim 14 wherein said solvent is selected from the group consisting of water, alcohols, alkanes, alkenes, ethers, ketones, polar aprotic and aromatic solvents.

19. The process of claim 1 wherein said process further comprises addition of a protic or a non-protic solvent to the ionic liquid.

20. The process of claim 1 wherein the operating temperature is between 0° C. and 100° C. and operating pressure is between 689 kPa (100 psi) and 14 MPa (2000 psi).
